(19) United States
(12) Patent Application Publication
(10) Pub. No.: US 2021/0290592 A1
(43) Pub. Date: Sep. 23, 2021

(54) COMPOSITION AND METHOD FOR OPIOID SPARING

(71) Applicant: Zelira Therapeutics Operations Pty Ltd., Perth, Western Australia (AU)

(72) Inventor: Harry KARELIS, Perth, Western Australia (AU)

(21) Appl. No.: 17/257,513

(22) PCT Filed: Jul. 3, 2019
(86) PCT No.: PCT/AU2019/050699

§ 371 Date: Dec. 31, 2020

(30) Foreign Application Priority Data
Jul. 3, 2018 (AU) 2018100928

(51) Int. Cl. A61K 31/352; A61K 31/05; A61K 36/185; A61K 31/01; A61K 31/015; A61K 31/045; A61K 31/485; A61K 31/4468; A61K 31/135; A61K 31/451; A61P 25/04; A61P 25/36

(52) U.S. Cl. CPC A61K 31/352; A61K 31/05; A61K 36/185; A61K 31/01; A61K 31/015; A61P 25/36; A61K 31/485; A61K 31/4468; A61K 31/135; A61K 31/451; A61P 25/04; A61K 31/045

(57) ABSTRACT

The invention relates to pharmaceutical compositions comprising Δ⁹-tetrahydrocannabinol (THC), cannabidiol (CBD) and a terpene fraction obtained by extraction of a Cannabis plant, and their use in the treatment of opioid dependence and/or addiction. The invention also relates to methods for opioid sparing.

### FIELD

The invention relates to pharmaceutical compositions comprising cannabidiol (CBD) and Δ⁹-tetrahydrocannabinol (THC), and their use as part of an opioid sparing treatment protocol. The invention also relates to methods for treating opioid dependence and/or addiction.

### BACKGROUND

Opioid based analgesia is commonly prescribed for both acute post-surgical pain and for chronic pain. In Australia, evidence-based guidelines support the use of opioids in the acute post-surgical setting but not for chronic non-cancer pain. There are numerous adverse effects of opioids including: over-sedation, which can lead to accidents and increased risk of falls, respiratory depression, sleep apnoea, nausea, vomiting, constipation, opioid-induced androgen deficiency and peripheral oedema. Other negative consequences of opioid use include unintentional overdose, the development of opioid dependence and diversion of opioids to individuals for non-medical purposes. Nevertheless, many patients with chronic pain (e.g. pain experienced daily for more than 3 months) are on long term treatment with high dose (OMEDD >60 mg) opioid analgesia.

Dose reduction is indicated where there is a pattern of escalating opioid use due to ineffective pain relief, where adverse effects are limiting quality of life, and where opioids are being misused. Generally, a fairly rapid dose reduction of between 10-25% per week is recommended. However, in cases where the patient has been on opioids for many years the dose reduction may be between 10-25% per month. Drop-out rates from dose taper regimens are high, some studies reporting drop-out rates between 75-100%.

There is some evidence that cannabinoids may enable patients to reduce their opioid medication whilst maintaining adequate analgesia—known as opioid sparing. If so, cannabinoids could prove effective in reducing drop-out rates in opioid dose taper regimen, assisting patients with poorly managed pain.

Lynch and Clark (Journal Pain Symptom Management. 2003; 25:496-498) reported the results of a clinical case series in which 3 patients with non-cancer pain reported regular use of smoked marijuana and were able to reduce their opioid dose by 60-100%.

Other studies have reported more mixed results. For example, Johnson et al. (Journal Pain Symptom Management. 2010; 39:167-179) reported that the efficacy of a cannabinoids in the oro-mucosal spray formulations (A: 2.7mg THC:2.5 mg CBD; B: THC only 2.7 mg, and C: placebo) in moderate to severe cancer related pain. In this two week double blind, randomized, placebo controlled, parallel group trial (n=177), patients could self-titrate their dose up to a maximum of 48 sprays per 24 hour period. Use of opioid medication was a secondary endpoint. No change in the median amount of opioid breakthrough medication was observed in any group.

Seeling et al. (Anaesthetist. 2006; 55:391-400) examined the use of dronabinol (5 mg THC) versus placebo in acute post-surgical pain. Median dose of micro-opioid agonist piritramide in the two days post surgery was 54 mg in the treatment group versus 74 mg in the control group; however, this difference was not statistically significant (n=105).

Despite the mixed results from previous studies, interest in the opioid sparing effects of cannabinoids remains high especially in the USA where dependence on prescribed opioids for chronic non-cancer pain conditions has been declared an epidemic. Very recently the NIH has provided $3.8 million to fund what is being described as the first long-term study to investigate whether treatment with medicinal cannabinoids can lead to a reduction in opioid use in adults with chronic pain (1R01DA044171-01A1). Over 18 months, 250 HIV-positive and HIV-negative adults with chronic pain who use opioids and who have received certification from their physicians to use medicinal cannabinoids, will complete fortnightly web-based questionnaires which will focus on pain levels and the medical and illicit use of marijuana and opioids. Urine and blood samples will be collected every three months and in-depth interviews will explore their perceptions of how medical marijuana use affects the use of opioids.

This US study will provide important information on how chronic pain sufferers are self-medicating with medical and illicit cannabinoids. However, this is not a clinical trial and will not provide quality data to inform guidelines needed now by clinicians to assist patients with chronic pain and problematic opioid use and/or dependence.

It would therefore be advantageous to provide pharmaceutical compositions comprising cannabinoids for use in an opioid sparing dosage regimen.

### SUMMARY

The inventors believe that treatment of patients suffering from non-cancer chronic pain with a pharmaceutical composition comprising Δ⁹-tetrahydrocannabinol (THC) and cannabidiol (CBD) and a synergistic terpene fraction may assist in an opioid sparing dosage regimen.

In one aspect, there is provided a pharmaceutical composition comprising THC, CBD and a terpene fraction obtained by extraction of a Cannabis plant. The pharmaceutical composition may optionally comprise one or more pharmaceutically acceptable excipient(s).

In another aspect, there is provided a method of opioid sparing in a subject in need thereof, the method comprising administering to the subject an effective amount of a pharmaceutical composition of the invention according to the following dosage regimen:
(1) administering an effective amount of an opioid together with an effective amount of the pharmaceutical composition of invention, and
(2) administering a sub-clinical amount of the opioid together with an effective amount of the pharmaceutical composition of the invention, and optionally
(3) administering an effective amount of the pharmaceutical composition of the invention without an opioid.

In yet another aspect, there is provided a method of treating opioid dependence and/or addiction in a subject in need thereof, the method comprising administering to the subject an effective amount of a pharmaceutical composition of the invention according to the following dosage regimen:
(1) administering an effective amount of an opioid together with an effective amount of the pharmaceutical composition of invention, and
(2) administering a sub-clinical amount of the opioid together with an effective amount of the pharmaceutical composition of the invention, and optionally
(3) administering an effective amount of the pharmaceutical composition of the invention without an opioid.

In some embodiments, step (2) of the dosage regimen in the methods above comprises reducing the dose of opioid by a tapering amount per week of treatment.

In a further aspect, there is provided a kit of parts comprising in separate parts: (a) an effective amount of a pharmaceutical composition of the invention, and (b) an opioid.

In still a further aspect, there is provided use of of one or more of (a) a Cannabis extract; (b) THC and (c) CBD in the manufacture of a medicament for opioid sparing in a subject in need thereof, wherein the medicament comprises THC, CBD and a terpene fraction.

In still a further aspect, there is provided use of of one or more of (a) a Cannabis extract; (b) THC and (c) CBD in the manufacture of a medicament for treating opioid dependence and/or addiction in a patient in need thereof, wherein the medicament comprises THC, CBD and a terpene fraction.

### DEFINITIONS

The term “cannabinoid” as used herein relates to any compound that has been isolated from a Cannabis plant or synthetically created that has activity involving the endocannabinoid system. The term is used to describe the relevant compound itself irrespective of its source.

The term “cannabinoid fraction” is used to describe the combination of cannabinoid compounds present in the Cannabis extract.

The term “terpenes” or “terpenoids” as used herein refers to a class of hydrocarbon molecules, which often provide a unique smell. Terpenes are derived from units of isoprene, which has the molecular formula C₅H₈. The basic molecular formula of terpenes are multiples of the isoprene unit, i.e. (C₅H₈)ₙ, where n is the number of linked isoprene units. Terpenoids are terpene compounds that have been further metabolised in the plant, typically through an oxidative process, and therefore usually contain at least one oxygen atom.

The term “terpene fraction” is used to describe the combination of terpene and terpenoid compounds present in the Cannabis extract.

As used herein, the terms “treating”, “treatment”, “treat” and the like mean affecting a subject, patient, tissue or cell to obtain a desired pharmacological and/or physiological effect. The effect may be prophylactic in terms of completely or partially preventing, or reducing the severity of, a disease or associated symptom, and/or may be therapeutic in terms of a partial or complete cure of a disease.

The term “administering” refers to providing the pharmaceutical composition to a patient suffering from or at risk of the disease(s) or condition(s) to be treated or prevented.

By “effective amount” it is meant an amount sufficient that, when administered to the patient, an amount of the drug is provided to achieve an effect. In the case of a therapeutic method, this effect may be the treatment of the specified disease and/or condition or a symptom thereof. Therefore, the “effective amount” may be a “therapeutically effective amount”. By “therapeutically effective amount” it is meant an amount sufficient that when administered to the patient an amount of active ingredient is provided to treat the disease or a symptom of the disease.

As used herein and in the appended claims, the singular forms “a,” “an,” and “the” include plural reference unless the context clearly dictates otherwise. Thus, for example, a reference to “an excipient” may include a plurality of excipients, and a reference to “a subject” may be a reference to one or more subjects, and so forth.

The term “(s)” following a noun contemplates the singular or plural form, or both.

The term “and/or” can mean “and” or “or”.

Unless the context requires otherwise, all percentages referred to herein are percentages by weight of the composition.

Various features of the invention are described and/or claimed with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, preferably means an amount within ±25%, ±10%, ±5%, ±1% or ±0.1% of that value.

The term “comprising” as used in this specification means “consisting at least in part of”. When interpreting statements in this specification that include that term, the features, prefaced by that term in each statement, all need to be present but other features can also be present. Related terms such as “comprise” and “comprised” are to be interpreted in the same manner.

Before describing the present invention in detail, it is to be understood that this invention is not limited to particularly exemplified pharmaceutical compositions, methods of production or treatment, which may, of course, vary. It is also to be understood that the terminology used herein is for the purpose of describing particular embodiments of the invention only, and is not intended to be limiting.

The inventions described and claimed herein have many attributes and embodiments including, but not limited to, those set forth or described or referenced in this summary section, which is not intended to be all-inclusive. The inventions described and claimed herein are not limited to or by the features or embodiments identified in this summary section, which is included for purposes of overview illustration only and not limitation.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

### DESCRIPTION OF EMBODIMENT(S)

The present invention provides a pharmaceutical composition comprising THC and CBD and a terpene fraction.

CBD is the main non-psychotropic phytocannabinoid present in the Cannabis sativa plant, in some cases constituting up to 40 per cent of its extract depending on extraction technique. Both animal and human studies suggest that the pharmacokinetics and pharmacodynamics of CBD are very complex. CBD appears to operate at both CB1 and CB2 endocannabinoid receptors within the endocannabinoid system (ECS) indirectly stimulating endogenous cannabinoid signaling (anadamine) by suppressing fatty acid amide hydrolase (FAAH), the enzyme that breaks down anandamide. Importantly, this enables more anandamide to remain at the receptors, which elicits anxiolytic and antidepressant like effects. This indirect agonist property at the cannabinoid receptors may also explain its promising safety profile. Furthermore, CBD has been shown to also act on the vanilloid, adenosine and serotonin receptors explaining its broad spectrum of potential therapeutic properties in animal models and humans, including anxiolytic, antidepressant, neuroprotective, anti-inflammatory and immunomodulatory actions.

THC is the main psychotropic constituent of Cannabis, its main pharmacological effects including analgesia, muscle relaxation, antiemesis, appetite stimulation and psychoactivity. THC mimics the action of the endogenous cannabinoid receptor ligands. THC is a partial agonist of CB1 receptors, which are primarily expressed in the central nervous system, especially in areas associated with pain. It is believed that THC induces analgesia by binding presynaptic CB1 receptors, inhibiting neurons activated by pain in these areas.

There is evidence that THC and CBD used in combination, act synergistically to maximize analgesic response. CBD has been demonstrated to antagonise some undesirable effects of THC including intoxication, sedation and tachycardia, while contributing analgesic, anti-emetic, and anti-carcinogenic properties.

The pharmaceutical composition may comprise THC and CBD in ratio of THC:CBD from about 10:1 to about 1:10. In some embodiments, the ratio of THC:CBD will be balanced, for example from about 2:1 to about 1:2, such as about 0.8:1 to about 1.2:1 or about 1:1.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The ratio of THC to CBD may be readily determined by methods known in the art, including High-Performance Liquid Chromatography (HPLC) and Ultra Performance Liquid Chromatography (UPLC).

In some embodiments, pharmaceutical compositions comprising THC may comprise THC in a minimum amount of at least about 15 wt %, for example, at least about 25 wt %, about 35 wt % or about 40 wt %. In some embodiments, the pharmaceutical composition comprises THC in a maximum amount of up to about 85 wt %, about 80 wt %, about 75 wt %, about 70 wt %, about 65 wt %, about 60 wt %, about 55 wt %, about 50 wt %, about 45 wt %, about 40 wt %, about 35 wt %, about 30 wt %, about 25 wt % or about 20 wt %. It will be appreciated that the amount of THC may be within the range from any of these minimum amounts to any of these maximum amounts. All combinations of these minimum and maximum amounts are contemplated. For example, in some embodiments, the pharmaceutical composition comprises THC in an amount of from about 15 wt % to about 85 wt %, about 15 wt % to about 75 wt %, about 15 wt % to about 40 wt % or about 40 wt % to about 60 wt %.

In some embodiments, pharmaceutical compositions comprising CBD may comprise CBD in a minimum amount of at least about 15 wt %, for example, at least about 25 wt %, about 35 wt % or about 40 wt %. In some embodiments, the pharmaceutical composition comprises CBD in a maximum amount of up to about 60 wt %, about 55 wt %, about 50 wt %, about 45 wt %, about 40 wt %, about 35 wt %, about 30 wt %, about 25 wt % or about 20 wt %. It will be appreciated that the amount of CBD may be within the range from any of these minimum amounts to any of these maximum amounts. All combinations of these minimum and maximum amounts are contemplated. For example, in some embodiments, the pharmaceutical composition comprises CBD in an amount of from about 15 wt % to about 60 wt %, about 15 wt % to about 55 wt %, about 15 wt % to about 40 wt % or about 40 wt % to about 55 wt %.

In some embodiments, the pharmaceutical composition comprises THC and CBD in a minimum total amount of at least about 30 wt %, for example, at least about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt % or about 99 wt %. In some embodiments, the pharmaceutical composition comprises THC and CBD in a total maximum amount of up to about 99 wt %, for example, up to about 95 wt %, about 90 wt %, about 85 wt %, about 80 wt %, about 70 wt %, about 60 wt %, about 50 wt %, about 40 wt %, about 30 wt %, about 20 wt % or about 15 wt %. It will be appreciated that the total amount of CBD and THC may be within the range from any of these minimum amounts to any of these maximum amounts. All combinations of these minimum and maximum amounts are contemplated. For example, in some embodiments, the pharmaceutical composition comprises CBD and THC in an amount of from about 30 wt % to about 99 wt %.

References to THC and CBD (and any other natural product, including cannabinoid(s), terpene(s) and terpenoid(s)) used herein include the relevant compound and pharmaceutically acceptable salts and/or solvates (including hydrates) thereof.

The THC and CBD may be combined from purified forms of the compounds, which may be purified after extraction from a natural source, or produced synthetically or semi-synthetically. Any means known in the art for producing CBD and/or THC is contemplated. Alternatively, the pharmaceutical composition may comprise a Cannabis extract comprising THC, CBD and a terpene fraction.

Cannabis plants produce a diverse array of secondary metabolites, including cannabinoids, terpenes, terpenoids, sterols, triglycerides, alkanes, squalenes, tocopherols, carotenoids and alkaloids. The mix of these secondary metabolites varies depending on several factors, including Cannabis variety, part of the Cannabis plant extracted, method of extraction, processing of the extract and season.

There are several varieties of Cannabis plant, which have been described under two distinct naming conventions. One of these conventions identifies three distinct species of Cannabis plant, namely Cannabis sativa Linnaeus, Cannabis indica LAM., and Cannabis ruderalis. Another convention identifies all Cannabis plants as belonging to the Cannabis sativa L. species, with the various varieties divided amongst several subspecies, including: Cannabis sativa ssp. sativa and ssp. indica. As used herein, the term “Cannabis” refers to any and all of these plant varieties.

Extracts of Cannabis may be prepared by any means known in the art. The extracts may be formed from any part of the Cannabis plant containing cannabinoid and terpene and/or terpenoid compounds. Extracts may be formed from a leaf, seed, trichome, flower, keif, shake, bud, stem or a combination thereof. The part of the Cannabis plant may be used fresh or dried prior to extraction. All known means of drying the plant material are contemplated. In some embodiments, the extract is formed by contacting any part of the Cannabis plant with an extractant. Any suitable extractant known in the art may be used, including, for example, alcohols (e.g. methanol, ethanol, propanol, butanol, propylene glycol etc.), water, hydrocarbons (e.g. butane, hexane, etc.), oils (e.g. olive oil, vegetable oil, essential oil, etc.), a polar organic solvent (e.g. ethyl acetate, polyethylene glycol, etc.) or a supercritical fluid (e.g. liquid CO₂). The extractant may be completely or partially removed prior to incorporation of the Cannabis extract into the pharmaceutical composition, or it may be included in the pharmaceutical composition as a carrier. The extractant may be removed by heating the extract optionally under reduced pressure (e.g. under vacuum). It will be appreciated that some of the more volatile plant metabolites (such as terpenes) may also be removed with the extractant. Accordingly, in some embodiments, removing the extractant may enrich the cannabinoid fraction of the extract. In some embodiments, the extract is filtered to remove particulate material, for example, by passing the extract through filter paper or a fine sieve (e.g. a sieve with pore sizes of 5 μm).

In some embodiments, the Cannabis extract is formed by applying heat and/or pressure to the plant material. Typically, in these embodiments, no extractant is required.

In some embodiments, one or more additional compounds (e.g. cannabinoid, terpene or terpenoid compounds) may be added to the Cannabis extract. The addition of compounds may be to compensate for natural variations in the relative amounts of certain compounds being expressed in the Cannabis plant. The added compounds may be synthetic versions of the desired compounds, they may be purified compounds obtained from other Cannabis extracts, or they may be added by blending two or more Cannabis extracts.

The cannabinoid fraction typically accounts for the majority of the compounds present in the Cannabis extract.

In some embodiments, the Cannabis extract may comprise about 35% to about 95% by weight cannabinoids, for example, about 40% to about 90%, about 45% to about 70% or about 45% to about 55% by weight of the Cannabis extract. In some embodiments, the Cannabis extract comprises about 5% to about 65% by weight of non-cannabinoids, for example, about 5% to about 50%, about 10% to about 40% by weight or about 15% to about 30% by weight non-cannabinoids.

The cannabinoid fraction of a Cannabis extract may comprise a primary (or main) cannabinoid. As used herein, the term “primary cannabinoid” relates to the cannabinoid present in a Cannabis extract is the highest concentration. Typically, the primary cannabinoid may be Δ⁹-Tetrahydrocannabinol (THC) or cannabidiol (CBD). The primary cannabinoid may be present in the Cannabis extract in an amount of at least about 0.1%, about 0.5%, about 1%, about 1.5%, about 2%, about 2.5%, about 3%, about 3.5%, about 4%, about 20%, about 25%, about 30%, about 35%, about 40%, about 45%, about 50% or about 55% by weight of the Cannabis extract. Accordingly, when THC is the primary cannabinoid, the Cannabis extract may comprise at least about 0.1%, about 0.5%, about 1%, about 1.5%, about 2%, about 2.5%, about 3%, about 3.5%, about 4%, about 4.5%, about 20%, about 25%, about 30%, about 35%, about 40%, about 45%, about 50% or about 55% by weight Δ⁹-tetrahydrocannabinol (THC), for example, about 0.1% to about 97%, about 0.1% to about 20%, or about 50 to about 90% by weight of Δ⁹-tetrahydrocannabinol (THC). When CBD is the primary cannabinoid, the Cannabis extract may comprise at least about 0.1%, about 0.5%, about 1%, about 1.5%, about 2%, about 2.5%, about 3%, about 3.5%, about 4%, about 20%, about 25%, about 30%, about 35%, about 40%, about 45%, about 50%, about 55% or about 60% by weight CBD, for example, about 0.1% to about 97%, about 0.1% to about 10% or about 50 to about 90% by weight CBD.

In addition to the primary cannabinoid, the Cannabis extract may further comprise a secondary cannabinoid. As used herein, the term “secondary cannabinoid” relates to the cannabinoid present in a Cannabis extract is the second highest concentration. The secondary cannabinoid is therefore present in the Cannabis extract in an amount less than the primary cannabinoid. In some embodiments where the primary cannabinoid is THC, the secondary cannabinoid may be CBD. In some embodiments where the primary cannabinoid is CBD, the secondary cannabinoid may be THC. The secondary cannabinoid may be present in the Cannabis extract in an amount of at least about 0.001% by weight, for example, at least about 0.005%, 0.01%, 0.05%, 0.1%, 0.5%, 1%, 1.5% or 2% by weight of the extract. The secondary cannabinoid may be present in a maximum amount of less than the amount of the primary cannabinoid, such as up to about 10%, for example, up to about 9%, 8%, 7%, 6%, 5% by weight of the extract. It will be appreciated that the amount of secondary cannabinoid may be within the range from any of these minimum amounts to any of these maximum amounts

In some embodiments, the Cannabis extract is enriched in one and/or the other of CBD or THC. It has been shown that endocannabinoids (i.e. naturally occurring cannabinoids), including CBD and THC, interact with a class of G protein-coupled receptors (GPCRs) named the “cannabinoid receptors”, e.g. the CB1 or CB2 receptors. However, structurally related cannabinoid compounds may have vastly different activity. Despite these differences in activity, the present invention relies on the activity of the combination of THC, CBD and the terpene fraction.

In some embodiments, the Cannabis extract may comprise at least about 0.001% by weight THC and/or CBD, for example, from about 0.001% to about 99.999% by weight THC and/or CBD, at least about 0.001% to about 20% by weight THC and/or CBD, about 0.01% to about 20% by weight THC and/or CBD, about 0.01% to about 15% by weight THC and/or CBD.

In some embodiments, the Cannabis extract may comprise THC and CBD in a combined weight of at least about 1% by weight, for example, at least about 5% by weight. In some embodiments, the combined amount of CBD and THC may be 1-20%, 1-15%, 6-11% or 50-90% by weight of the pharmaceutical composition. The ratio of THC to CBD may be from about 100:0 to about 0:100, about 100:1 to about 1:100, about 80:1 to about 1:80, about 60:1 to about 1:60, about 40:1 to about 1:40 or about 20:1 to about 1:20. In some embodiments, the ratio of THC to CBD may be balanced, for example in a ratio of THC:CBD of about 2:1 to about 1:2 or about 1:1. The ratio of THC:CBD may be expressed as a single number by dividing the amount of THC by the amount of CBD present. Accordingly, the ratio of THC:CBD in the pharmaceutical compositions may be 0.001, 0.1, 0.2, 0.3, 0.4, 0.45, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, or higher. In some embodiments, the ratio of THC:CBD in a Cannabis extract may be between any of these values, for example, from 0.001 to 3, 0.2 to 3 or 0.4 to 2.6.

Embodiments of the pharmaceutical composition comprising a balanced amount of THC and CBD may be obtained by, for example, adding CBD to a Cannabis extract that comprises THC as primary cannabinoid, adding THC to a Cannabis extract that comprises CBD as primary cannabinoid, or combining a Cannabis extract comprising THC as primary cannabinoid with a Cannabis extract comprising CBD as primary cannabinoid.

Embodiments of the pharmaceutical composition enriched in one or the other of THC or CBD may be obtained by, for example, adding purified or synthetic THC or CBD to a Cannabis extract, to obtain the desired amount of THC or CBD or the desired ratio of THC to CBD.

Typically, the Cannabis extract may also comprise other cannabinoids in addition to THC and/or CBD, such as any of the cannabinoids previously identified in Cannabis plants. To date, over 100 cannabinoids have been identified in Cannabis plants. A comprehensive list of these cannabinoids may be found in Mahmoud A. El Sohly and Waseem Gul, “Constituents of Cannabis Sativa.” In Handbook of Cannabis Roger Pertwee (Ed.) Oxford University Press (2014) (ISBN: 9780199662685). Cannabinoids that have been identified in Cannabis plants include: Cannabigerol (E)-CBG-C5, Cannabigerol monomethyl ether (E)-CBGM-C5 A, Cannabigerolic acid A (Z)-CBGA-C5 A, Cannabigerovarin (E)-CBGV-C3, Cannabigerolic acid A (E)-CBGA-C5 A, Cannabigerolic acid A monomethyl ether (E)CBGAM-C5 A and Cannabigerovarinic acid A (E)-CBGVAC3A; (±)-Cannabichromene CBC-C5, (±)-Cannabichromenic acid A CBCA-C5 A, (±)-Cannabivarichromene, (±)-Cannabichromevarin CBCV-C3, (±)-Cannabichromevarinic acid A CBCVA-C3 A; (−)-Cannabidiol CBD-C5, Cannabidiol momomethyl ether CBDMC5, Cannabidiol-C4 CBD-C4, (−)-Cannabidivarin CBDVC3, Cannabidiorcol CBD-Cl, Cannabidiolic acid CBDA-C5, Cannabidivarinic acid CBDVA-C3; Cannabinodiol CBNDC5, Cannabinodivarin CBND-C3; Δ⁹-Tetrahydrocannabinol Δ⁹-THC-C5, Δ⁹-Tetrahydrocannabinol-C4 Δ⁹-THCC4, Δ⁹-Tetrahydrocannabivarin Δ⁹-THCV-C3, Δ⁹-Tetrahydrocannabiorcol Δ⁹ THCO Cl, Δ⁹-Tetrahydrocannabinolic acid Δ⁹-THCA-C5 A, Δ⁹-Tetrahydrocannabinolic acid B Δ⁹-THCA-C5 B, Δ⁹-Tetrahydrocannabinolic acid-C4 A and/or B Δ⁹-THCA-C4 A and/or B, Δ⁹-Tetrahydro-cannabivarinic acid A Δ⁹-THCVA-C3 A, Δ⁹-Tetrahydrocannabiorcolic acid A and/or B Δ⁹-THCOA-Cl A and/or B), (−)-Δ⁸-trans-(6aR,10aR)-Δ⁸-Tetrahydrocannabinol Δ⁸-THC-C5, (−)-Δ⁸-trans-(6aR,10aR)-Tetrahydrocannabinolic acid A Δ⁸-THCA-C5 A, (−)-(6aS,10aR)-Δ⁹-Tetrahydrocannabinol (−)-cis-Δ⁹-THC-C5; Cannabinol CBN-C5, Cannabinol-C4 CBN-C4, Cannabivarin CBN-C3, Cannabinol C2 CBN-C2, Cannabiorcol CBN-Cl, Cannabinolic acid A CBNA-C5 A, Cannabinol methyl ether CBNM-C5, (−)-(9R,10R)-trans-Cannabitriol (−)-trans-CBT-C5, (+)-(9S,10S)-Cannabitriol (+)-trans-CBT-C5, (±)-(9R,10S/9S,10R)—); Cannabitriol (±)-cis-CBT-C5, (−)-(9R,10R)-trans-10-O-Ethyl-cannabitriol (−)-trans-CBT-OEt-C5, (±)-(9R,10R/9S,10S)-Cannabitriol-C3 (±)-trans-CBT-C3, 8,9-Dihydroxy-Δ6a(10a)-tetrahydrocannabinol 8,9-Di-OH-CBT-C5, Cannabidiolic acid A cannabitriol ester CBDA-C5 9-OH-CBT-C5 ester, (−)-(6aR,9S,10S,10aR)-9,10-Dihydroxyhexahydrocannabinol, Cannabiripsol, Cannabiripsol-C5, (−)-6a,7,10a-Trihydroxy-Δ⁹-tetrahydrocannabinol (−)-Cannabitetrol, 10-Oxo-Δ6a(10a)tetrahydrocannabinol OTHC); (5aS,6S,9R,9aR)-Cannabielsoin CBE-C5, (5aS,6S,9R,9aR)-C3-Cannabielsoin CBE-C3, (5aS,6S,9R,9aR)-Cannabielsoic acid A CBEA-C5 A, (5aS,6S,9R,9aR)-Cannabielsoic acid B CBEA-C5 B; (5aS,6S,9R,9aR)-C3-Cannabielsoic acid B CBEA-C3 B, Cannabiglendol-C3 OH-iso-HHCV-C3, Dehydrocannabifuran DCBF-C5, Cannabifuran CBF-C5), (−)-Δ⁷-trans-(1R,3R,6R)-Isotetrahydrocannabinol, (±)-Δ⁷-1,2-cis-(1R,3R,6S/1S,3S,6R)-Isotetrahydrocannabivarin, (−)-Δ⁷-trans-(1R,3R,6R)-Isotetrahydrocannabivarin; (±)-(IaS,3aR,8bR,8cR)-Cannabicyclol CBL-C5, (±)-(1aS,3aR,8bR,8cR)-Cannabicyclolic acid A CBLA-C5 A, (±)-(IaS,3aR,8bR,8cR)-Cannabicyclovarin CBLV-C3; Cannabicitran CBTC5; Cannabichromanone CBCN-C5, CannabichromanoneC3 CBCN-C3, and Cannabicoumaronone CBCON-C5.

In some embodiments, the Cannabis extract further comprises one or more of Δ⁹-Tetrahydrocannabinolic acid (THCA), Δ⁹-Tetrahydrocannabivann (THCV), (−)-Cannabidivarin (CBDV), Cannabinodiol (CBN), Cannabichromene (CBC) and Cannabigerol (CBG). Each of these cannabinoids may be present in an amount from about 0.001% to about 40% by weight of the Cannabis extract. Typically, the other cannabinoids are present in amounts lower than the primary cannabinoid or, if present, the secondary cannabinoid(s).

In some embodiments, certain cannabinoids may be absent, or present in non-detectable amounts (e.g. less than about 0.001% by weight of the analyte). In some embodiments, the Cannabis extract may exclude one or more of the following cannabinoids: Δ⁹-Tetrahydrocannabinolic acid (THCA), Δ⁹-Tetrahydrocannabivann (THCV), Cannabidiolic acid (CBDA), Cannabinodiol (CBN), (−)-Cannabidivarin (CBDV), Cannabigerol (CBG) and Cannabichromene (CBC).

Cannabis extracts may further comprise a non-cannabinoid fraction. The non-cannabinoid fraction may include a terpene fraction. In some embodiments, the Cannabis extract comprises a terpene fraction in an amount of less than about 50% by weight, for example, less than about 45%, about 40%, about 35%, about 30%, about 25%, about 20%, about 15%, about 10%, about 9%, about 8%, about 7%, about 6%, about 5%, about 4%, about 3%, about 2% or about 1% by weight of the extract. In some embodiments, the Cannabis extract may comprise terpene and terpenoid compounds in an amount of at least about 0.001% by weight of the extract, for example, at least about 0.005%, about 0.01%, about 0.05%, about 0.1%, about 0.2%, about 0.25%, about 0.3%, about 0.35%, about 0.4%, about 0.45%, about 0.5%, about 1%, about 2%, about 3%, about 4%, about 5%, about 6%, about 10%, about 15% or more of the total weight of the extract. In some embodiments, the pharmaceutical composition comprises about 0.001% to about 50% by weight of terpene and terpenoid compounds, for example, about 0.01% to about 50% by weight, about 0.01% to about 10% by weight, about 0.01% to about 6% by weight or about 0.01 to about 5% by weight of the pharmaceutical composition.

Typically, the terpene fraction in the plant material used to form the extract may have a different terpene/terpenoid profile than the terpene profile of the final extract, both in terms of the amounts of specific compounds in the terpene fraction and the weight of the terpene fraction relative to the other components. For example, a Cannabis flower may comprise about 20% by weight cannabinoids and about 3% by weight terpenes. Following extraction and concentration (i.e. removal of the extractant), the cannabinoid fraction may amount to about 50-90% by weight and the terpene fraction may amount to about 0.1-6% by weight of the Cannabis extract. This typical scenario shows that the cannabinoids are concentrated when the extractant is removed, the relative amount of the terpene fraction is reduced, likely due to the volatility of many of the terpenes/terpenoids present in the terpene fraction. Therefore, the profile of the terpene fraction present in the Cannabis extract is significantly different from the profile of the terpene fraction that exists in Nature.

The efficacy of a composition may be enhanced when the terpene fraction has a certain profile, i.e. a certain proportion of particular terpenes/terpenoids are present in the extract. It is believed that the increase in efficacy may be synergistic (i.e. non-additive). It is also believed that the presence of specific components in the terpene fraction may enhance the patient's tolerance to cannabinoid therapy.

A variety of terpenes and terpenoids have also been identified in Cannabis extracts, including monoterpenes, monoterpenoids, sesquiterpenes and sesquiterpenoids. For example, the following terpenes and terpenoids have been identified in Cannabis extracts: Alloaromadendrene, allyl hexanoate, benzaldehyde, (Z)-a-cis-bergamotene, (Z)-a-trans-bergamotene, β-bisabolol, epi-α-bisabolol, β-bisabolene, borneol (camphol), cis-γ-bisabolene, bomeol acetate (bomyl acetate), α-cadinene, camphene, camphor, cis-carveol, caryophyllene (β-caryophyllene), α-humulene (α-caryophyllene), γ-cadinene, Δ-3-carene, caryophyllene oxide, 1,8-cineole, citral A, citral B, cinnameldehyde, α-copaene (aglaiene), γ-curcumene, β-cymene, β-elemene, γ-elemene, ethyl decadienoate, ethyl maltol, ethyl propionate, ethylvanillin, eucalyptol, α-eudesmol, β-eudesmol, γ-eudesmol, eugenol, cis-β-famesene ((Z)-β-farnesene), trans-α-farnesene, trans-β-famesene, trans-γ-bisabolene, fenchone, fenchol (norbomanol, β-fenchol), geraniol, α-guaiene, guaiol, gurjunene methyl anthranilate, methyl salicylate, 2-methyl-4-heptanone, 3-methyl-4-heptanone, hexyl acetate, ipsdienol, isoamyl acetate, lemenol, limonene, d-limonene (limonene), linolool (linalyl alcohol, β-linolool), α-longipinene, menthol, γ-muurolene, myrcene (β-myrcene), nerolidol, trans-nerolidol, nerol, β-ocimene (cis-ocimene), octyl acetate, α-phellandrene, phytol, α-pinene (2-pinene), β-pinene, pulegone, sabinene, cis-sabinene hydrate (cis-thujanol), β-selinene, α-selinene, γ-terpinene, terpinolene (isoterpine), terpineol (α-terpineol), terpineol-4-ol, α-terpinene (terpilene), α-thujene (origanene), vanillin, viridiflorene (ledene), and α-ylange. In some embodiments, the pharmaceutical composition comprises one or more of these terpenes and/or terpenoids.

In some embodiments, the Cannabis extract may comprise one or more of β-myrcene, α-terpinene, linalool, α-phellandrene, camphene, terpinolene, p-cymene, 1,8-cineole, β-caryophyllene, d-limonene, γ-terpinene, α-pinene, guaiol, gurjunene, β-ocimene, β-pinene, γ-cadinene, caryophyllene oxide, nerolidol and β-farnesene. For example, the Cannabis extract may comprise one, two, three, four, five or more of these terpenes/terpenoids. Each of these terpenoids may be absent or may be present in an amount in the range of 0.001% to 50% by weight of the terpene fraction.

In some embodiments, the terpene fraction comprises at least one of β-myrcene, α-terpinene, linalool, α-phellandrene, camphene, terpinolene, p-cymene, 1,8-cineole, β-caryophyllene, d-limonene, γ-terpinene, α-pinene and guaiol, especially at least two, at least three or at least four of these terpene/terpenoids.

In some embodiments, the terpene fraction comprises at least one of β-myrcene, α-terpinene, linalool, α-phellandrene, camphene, terpinolene, p-cymene, 1,8-cineole and β-caryophyllene, especially at least two, at least three or at least four of these terpene/terpenoids.

In some embodiments, the terpene fraction comprises at least one of β-myrcene, α-terpinene, linalool and α-phellandrene, especially two, three or four of these terpenes. In some embodiments the terpene fraction comprises all of β-myrcene, α-terpinene, linalool and α-phellandrene.

In some embodiments, the terpene fraction comprises at least one of the combinations β-myrcene and α-terpinene; β-myrcene and linalool; β-myrcene and α-phellandrene; α-terpinene and linalool; α-terpinene and α-phellandrene; linalool and α-phellandrene; β-myrcene, α-terpinene and linalool; β-myrcene, α-terpinene and α-phellandrene; β-myrcene, linalool and α-phellandrene; α-terpinene, linalool and α-phellandrene; and β-myrcene, α-terpinene, linalool and α-phellandrene or any of the above combinaions with one or more terpene/terpenoids selected from camphene, terpinolene, p-cymene, 1,8-cineole and β-caryophyllene.

In some embodiments, specific terpenes or terpenoids may be absent, or present in non-detectable amounts (e.g. less than about 0.001% by weight of the analyte).

The identity and amounts of terpenes and/or terpenoids obtained by extraction of a Cannabis plant may be determined by methods known in the art, including gas chromatography (GC). Typically, the profile of a cannabinoid fraction and a terpene fraction of a Cannabis extract are determined separately using different analytical techniques.

The pharmaceutical composition comprises THC, CBD and a terpene fraction. In some embodiments, the pharmaceutical composition consists of a Cannabis extract and optionally one or more pharmaceutically acceptable excipients, such as a carrier. In some embodiments, the pharmaceutical composition comprises a Cannabis extract to which has been added one or more of THC, CBD, one or more terpenes and/or terpenoids. The addition of compounds may be to compensate for natural variations in the relative amounts of certain compounds being expressed in the Cannabis plant or may be to enhance the activity of one or more cannabinoid, terpene or terpenoid compounds present in the extract or to provide the desired amount of the compound that is added. Terpenes and/or terpenoids may be added to adjust their content in the pharmaceutical composition to compensate for loss during an extraction process or to provide a desired non-natural terpene/terpenoid content in the pharmaceutical composition. The added compounds may be synthetic versions of the desired compounds, they may be purified compounds obtained from other Cannabis extracts or other plant extracts, or they may be added by blending two or more Cannabis extracts.

In some embodiments, the pharmaceutical composition optionally comprises one or more pharmaceutically acceptable excipient(s). The excipient may be a carrier, diluent, adjuvant, or other excipient, or any combination thereof, and “pharmaceutically acceptable” meaning that they are compatible with the other ingredients of the pharmaceutical composition and are not deleterious to a patient upon or following administration. The pharmaceutical compositions may be formulated, for example, by employing conventional solid or liquid vehicles or diluents, as well as pharmaceutical additives of a type appropriate to the mode of desired administration (for example, excipients, binders, preservatives, stabilisers, flavours, etc.) according to techniques such as those well known in the art of pharmaceutical formulation (See, for example, Remington: The Science and Practice of Pharmacy, 21st Ed., 2005, Lippincott Williams & Wilkins). The pharmaceutically acceptable carrier may be any carrier included in the United States Pharmacopeia/National Formulary (USP/NF), the British Pharmacopoeia (BP), the European Pharmacopoeia (EP), or the Japanese Pharmacopoeia (JP). In some embodiments, the excipient may be non-natural (e.g. synthetically produced).

The pharmaceutical composition includes those suitable for oral, rectal, nasal, topical (including oro-mucosal such as buccal and sublingual), vaginal or parenteral (including intramuscular, sub-cutaneous and intravenous) administration or in a form suitable for administration by inhalation or insufflation.

The ingredients of the pharmaceutical composition may be placed into the form of pharmaceutical compositions and unit dosages thereof, and in such form may be employed as solids, such as tablets or filled capsules, or liquids such as solutions, suspensions, emulsions, elixirs, or capsules or syringes filled with the same, all for oral use, in the form of suppositories for rectal administration; or in the form of sterile injectable solutions for parenteral (including subcutaneous) use.

Such pharmaceutical compositions and unit dosage forms thereof may comprise conventional ingredients in conventional proportions, with or without additional active ingredient(s), and such unit dosage forms may contain any suitable effective amount of the active ingredients commensurate with the intended daily dosage range to be employed.

For preparing pharmaceutical compositions described herein, pharmaceutically acceptable carriers can be either solid or liquid. Solid form preparations include powders, tablets, pills, capsules, cachets, suppositories, and dispensable granules. A solid carrier can be one or more substances which may also act as diluents, flavouring agents, solubilisers, lubricants, suspending agents, binders, preservatives, tablet disintegrating agents, or an encapsulating material.

Suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Tablets, powders, capsules, pills, cachets, and lozenges can be used as solid forms suitable for oral administration.

Liquid form preparations include solutions, dispersions, suspensions, and emulsions, for example, water or water-propylene glycol solutions or in oils such as vegetable oils. For example, parenteral injection liquid preparations can be formulated as solutions in aqueous polyethylene glycol solution. Liquid preparations are preferred for embodiments involving sublingual administration.

In some embodiments, the pharmaceutical composition is formulated for sublingual or buccal administration. Typically, a sublingual or buccal pharmaceutical composition is a liquid; however, any other suitable dosage form known in the art may be employed including aerosols, lozenges, troches, films, foams, pastes and dissolvable tablets.

Sterile liquid form pharmaceutical compositions include sterile solutions, suspensions, emulsions, syrups and elixirs. The active ingredient(s) may be suspended in a pharmaceutically acceptable carrier, such as sterile water, sterile organic solvent or a mixture of both.

Other liquid form preparations include those prepared by combining the Cannabis extract with one or more naturally derived oils (e.g. an essential oil) or waxes. An “essential oil” is an oil derived by extraction (e.g. steam extraction, or contacting the plant material with an extractant) or pressing, which contains primarily hydrophobic, and generally fragrant, components of the plant material. Suitable naturally derived oils and waxes include Sesame oil, Olive oil, Arnica essential oil, Lavender essential oil, Lavender Spike essential oil, Frankincense essential oil, Lemongrass essential oil, Cinnamon Leaf essential oil, Rosemary Cineole essential oil, Rosemary essential oil, Bergamot essential oil, Myrrh essential oil, Sage essential oil, Coconut oil, Bees wax and Hemp oil.

The pharmaceutical compositions may be formulated for parenteral administration (e. g. by injection, for example bolus injection or continuous infusion) and may be presented in unit dose form in ampoules, pre-filled syringes, small volume infusion or in multi-dose containers optionally with an added preservative. The pharmaceutical compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical forms suitable for injectable use include sterile injectable solutions or dispersions, and sterile powders for the extemporaneous preparation of sterile injectable solutions. They should be stable under the conditions of manufacture and storage and may be preserved against oxidation and the contaminating action of microorganisms such as bacteria or fungi.

The solvent or dispersion medium for the injectable solution or dispersion may contain any of the conventional solvent or carrier systems, and may contain, for example, water, ethanol, polyol (for example, glycerol, propylene glycol and liquid polyethylene glycol, and the like), suitable mixtures thereof, and vegetable oils.

Pharmaceutical forms suitable for injectable use may be delivered by any appropriate route including intravenous, intramuscular, intracerebral, intrathecal, epidural injection or infusion.

Sterile injectable solutions are prepared by incorporating the active ingredients in the required amount in the appropriate carrier with various other ingredients such as those enumerated above, as required, followed by sterilisation. Generally, dispersions are prepared by incorporating the various sterilised active ingredients into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile suspension of the active ingredient plus any additional desired ingredients.

For oral administration, the active ingredient(s) may be incorporated with excipients and used in the form of ingestible tablets, buccal tablets, troches, capsules, elixirs, suspensions, syrups, wafers, and the like.

The amount of active ingredient(s) in a therapeutically useful pharmaceutical composition should be sufficient that a suitable dosage will be obtained. Accordingly, the active ingredient(s) are preferably provided in an effective amount.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient(s), sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient(s) may be incorporated into sustained-release preparations and formulations, including those that allow specific delivery of the active peptide to specific regions of the gut.

Aqueous solutions can be prepared by dissolving the active ingredient(s) in water and adding suitable colorants, flavours, stabilising and thickening agents, as desired. Aqueous suspensions can be made by dispersing the finely divided active ingredient(s) in water with viscous material, such as natural or synthetic gums, resins, methylcellulose, sodium carboxymethylcellulose, or other well-known suspending agents.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations for oral and/or sublingual administration. Such liquid forms include solutions, suspensions, and emulsions. These preparations may contain, in addition to the active ingredient(s), colorants, flavours, stabilisers, buffers, artificial and natural sweeteners, dispersants, thickeners, solubilising agents, and the like.

For topical administration to the epidermis the active ingredient(s) may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth (oromucosal e.g. sublingual or buccal administration) include any liquid formulation described herein, preferably liquid formulations with a viscosity suitable for administration by dropper or syringe; lozenges comprising active ingredient(s) in a flavoured base, usually sucrose and acacia or tragacanth; pastilles comprising the active ingredient(s) in an inert base such as gelatin and glycerin or sucrose and acacia; and mouthwashes comprising the active ingredient(s) in a suitable liquid carrier.

For administration to the nasal cavity, solutions or suspensions may be applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension.

In the case of a spray, this may be achieved for example by means of a metering atomising spray pump. For such sprays, active ingredient(s) may be encapsulated with cyclodextrins, or formulated with other agents expected to enhance delivery and retention in the nasal mucosa.

Administration to the respiratory tract may be achieved by means of an aerosol formulation in which the active ingredient(s) are provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant. The dose of drug may be controlled by provision of a metered valve.

Alternatively, the active ingredient(s) may be provided in the form of a dry powder, for example a powder mix of the active ingredient(s) in a suitable powder base such as lactose, starch, starch derivatives such as hydroxypropylmethyl cellulose and polyvinylpyrrolidone (PVP). The pharmaceutical composition as a powder may be presented in unit dose form for example in capsules or cartridges of, e.g. gelatin, or blister packs from which the powder may be administered by means of an inhaler.

In formulations intended for administration to the respiratory tract, including intranasal formulations, the pharmaceutical composition may have a small particle size for example of the order of 5 to 10 microns or less. Such a particle size may be obtained by means known in the art, for example by micronisation.

When desired, formulations adapted to give sustained release of the active ingredient(s) may be employed.

The pharmaceutical composition may be prepared in unit dosage form. In such form, the composition is subdivided into unit doses containing appropriate quantities of the active ingredient(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Pharmaceutical compositions for parenteral administration may also be provided in unit dosage form for ease of administration and uniformity of dosage. Unit dosage form as used herein refers to physically discrete units suited as unitary dosages for the patients to be treated; each unit containing a predetermined quantity of active material calculated to produce the desired therapeutic effect in association with the required pharmaceutical excipient. The specification for the unit dosage forms are dictated by and directly dependent on (a) the unique characteristics of the active ingredient(s) and the particular therapeutic effect to be achieved, and (b) the limitations inherent in the art of compounding such an active ingredient(s) for the treatment of living patients having a diseased condition in which bodily health is impaired.

In some embodiments, the pharmaceutical composition comprises a further active ingredient. In some embodiments, the pharmaceutical composition comprises a further active ingredient other than a cannabinoid and/or terpene. Any suitable further active ingredient may be used provided that the activity of the active ingredient, THC, CBD and the terpene fraction is not diminished when combined. In some embodiments, the further active ingredient is an analgesic or antinoiciceptive drug or an opioid antagonist. In some embodiments, the analgesic or antinoiciceptive drug is a non-opioid alangesic or antinoiciceptive drug. Suitable non-opioid analgesic or antinoiceceptive drugs include fatty acid amide hydrolase (FAAH) inhibitors (such as paracetamol), non-steroidal antiinflammatory drugs (NSAIDs) (such as ibuprofen, aspirin and naproxen), COX-2 inhibitors (such as refecoxib, celecoxib and etoricoxib), antidepressants (such as amitriptyline, duloxetine, hydroxyzine, promethazine, carisoprodol, tripelennamine, clomipramine, amitriptyline), adjuvant analgesics (such as nefopam, orphenadrine, pregabalin, cyclobenzaprine, hycosine), anticonvulsants (such as carbamazepine, gabapentin), non-opioid NMDA antagonists (such as piritamide and flupiritine), stimulants (such as methylphenidate, caffeine, ephedrine, dextroamphetamine, methamphetamine, pseudoephedrine, phenylephrine and cocaine), and combinations thereof. Suitable opioid antagonists include naloxone, naltrexone, nalmefene, nalorphine, nalorphine dinicotinate, levallorphan, samidorphan, cyprodime, naltrindole, norbinaltorphimine, J-113,397, AT-076 and combinations thereof.

In some embodiments, the further active ingredient is an opioid. These embodiments may be useful during the early phases of treatment, or during the tapering phase of the therapy. Suitable opioids include morphinan opioids and non-morphinan opioids, for example, oxycodone, hydrocodone, oxymorphone, morphine, codeine, fentanyl, buprenorphine, tramadol, pethidine, and combinations thereof. The pharmaceutical composition may comprise an opioid in an effective amount, or in a sub-clinical amount.

The practice of the present invention employs, unless otherwise indicated, conventional pharmaceutical, veterinary and medical techniques within the skill of the art. Such techniques are well known to the skilled worker, and are explained fully in the literature.

### Methods of Treatment

The present invention provides a method for treating opioid dependence and/or addiction, comprising administering to a patient in need thereof an effective amount of a pharmaceutical composition of the invention.

The present invention also provides a method for opioid sparing, comprising administering to a patient in need thereof an effective amount of a pharmaceutical composition of the invention.

The present invention also provides a method for treating chronic non-cancer pain, comprising administering to a patient in need thereof an effective amount of a pharmaceutical composition of the invention.

Any pharmaceutical composition described herein may be used in these methods.

The methods of the invention may involve the following dosage regimen:
(1) administering an effective amount of an opioid together with an effective amount of the pharmaceutical composition of invention,
(2) administering a sub-clinical amount of the opioid together with an effective amount of the pharmaceutical composition of the invention, and optionally
(3) administering an effective amount of the pharmaceutical composition of the invention without an opioid.

Step (1) of the dosage regimen may be maintained for a period of up to about 10 weeks, for example, from about 1 week to about 10 weeks, about 1 week to about 5 weeks or for about 1 week only.

Step (2) of the dosage regimen may be maintained for a period of up to 20 weeks, for example, from 1 week to about 20 weeks, about 5 weeks to about 15 weeks or about 10 weeks. In some embodiments, step (2) involves reducing the dose of opioid by a tapering amount of about 1% to about 50% per week of treatment, for example, about 5% to about 20%, about 5% to about 15% per week or about 10% per week of treatment.

Step (3) of the dosage regimen is optional. Accordingly, in some embodiments, administration of the cannabinoid is stopped when administration of the opioid is stopped (e.g. when the dosage of opioid is reduced to zero). When included in the methods of the invention, step (3) may be maintained for as long a period as required. In some embodiments, step (3) is maintained for at least 1, 2, 3, 4, 5, 6, 7, 8, 9, 10, 11, 12, 13, 14, 15, 16 weeks of more, for example, step (3) may be maintained for about 1 to about 52 weeks, about 2 to about 20 weeks or about 6 weeks.

The method may further comprise, prior to step (1), administering an effective amount of an opioid without a cannabinoid (e.g. THC or CBD) to the subject. This step (prior to step (1) of the dosage regimen) may be maintained for up to 1 year (i.e. 52 weeks) or longer, for example, at least about 4 weeks, 8 weeks, 16 weeks, 24 weeks, 26 weeks, 42 weeks, 50 weeks, 52 weeks, 1.5 years, 2 years, 3 years, 4 years, 5 years, 10 years or longer.

The method may further comprise administering an increased dose of the opioid or the pharmaceutical composition of the invention to manage breakout pain experienced by the subject.

In some embodiments, the dosage of THC administered to the subject may be from about 5 mg to about 100 mg per day, for example, from about 10 mg to about 90 mg, about 30 mg to about 60 mg or about 50 mg per day.

In some embodiments, the dosage of CBD administered to the subject may be from about 5 mg to about 100 mg per day, for example, from about 10 mg to about 90 mg, about 30 mg to about 60 mg or about 50 mg per day.

The dosage of the terpene fraction will typically be about 0.01 wt % to about 20 wt % or about 0.1 wt % to about 6 wt % based on the amount of THC or CBD, whichever is greater.

The effective amount of the pharmaceutical composition of the invention may be held constant throughout the dosage regimen, or it may be altered depending on the symptoms of the subject. In some embodiments, the method further comprises a step of titrating the dose of the pharmaceutical composition for an individual subject.

In some embodiments, the pharmaceutical composition may be administered 1, 2, 3, 4 or more times per day. In order to facilitate the desired dosing schedule, the pharmaceutical composition may be formulated with a convenient concentration of active ingredient(s).

The method may comprise administering more than one pharmaceutical composition of the present invention to the patient in need thereof. For example, in some circumstances, it is preferred to administer a pharmaceutical composition high in THC and a pharmaceutical composition high in CBD to the patient. These pharmaceutical compositions may be administered in an alternating order and separated by a period of time. For example, the administration of high-THC and high-CBD formulations may be on alternating days, alternating sequences of days, or alternating from a high-CBD formulation in the morning to a high-THC formulation at night.

The method may also comprise administering any of the further active ingredient(s) described above. This active ingredient may be administered simultaneously, separately or consecutively with pharmaceutical compositions of the invention. By simultaneously it is meant that each of pharmaceutical composition and the other active ingredient are administered at the same time either in the same pharmaceutical composition or in separate pharmaceutical compositions. By separately it is mean that each of pharmaceutical composition and the other active ingredient are administered at the same time in different pharmaceutical compositions and optionally by different routes of administration. By consecutively it is meant that each of pharmaceutical composition and the other active ingredient are administered separately and may be at different times. Typically, when the pharmaceutical composition and the other active ingredient are administered consecutively they are administered within 24 hours, or within 12, 8, 6, 5, 4, 3, 2, or 1 hour(s) of each other. The pharmaceutical composition may be administered before or after the other active ingredient. Further, the route of administration for the pharmaceutical composition and the other active ingredient may be the same or different.

The pharmaceutical composition may be administered by any suitable route of administration.

The present invention also provides use of a Cannabis extract in the manufacture of a medicament for treating opioid dependence and/or addiction, wherein the medicament comprising THC, CBD and a terpene fraction. The Cannabis extract comprises at least the terpene fraction obtained by extraction of a Cannabis plant. In some embodiments, the Cannabis extract also comprises THC and CBD; however, where one or both of these cannabinoids are absent from the Cannabis extract used in the manufacture of the medicament, they may be added from another source, for example, from a synthetic source or from one or more further Cannabis extracts. The Cannabis extract may be obtained by any of the methods described above.

Also provided is the use of THC in the manufacture of a medicament for treating opioid dependence and/or addiction, wherein the medicament comprising THC, CBD and a terpene fraction. Also provided is the use of CBD in the manufacture of a medicament for treating opioid dependence and/or addiction, wherein the medicament comprising THC, CBD and a terpene fraction.

Also provided is a pharmaceutical composition comprising THC, CBD and a terpene fraction obtained by extraction of a Cannabis plant for treating opioid dependence and/or addiction, for opioid sparing or for treating chronic non-cancer pain.

Also provided is an opioid sparing agent comprising THC, CBD and a terpene fraction obtained by extraction of a Cannabis plant. The opioid sparing agent may comprise any of the pharmaceutical compositions described herein in an effective amount for reducing the amount of opioid required to manage pain in a subject in need thereof. In some embodiments, the opioid sparing agent comprises a therapeutically effective amount of THC, CBD and a terpene fraction obtained by extraction of a Cannabis plant.

The present invention further provides a kit comprising in separate parts: (A) THC and (B) CBD, wherein at least one of the parts (A) and/or (B) further comprise a terpene fraction obtained from extraction of a Cannabis plant.

The present invention further provides a kit comprising in separate parts: (a) an effective amount of a pharmaceutical composition of the invention, and (b) an opioid. The opioid may be included in an effective amount. In some embodiments, opioid may be included in a sub-clinical amount. Preferably, the kit may comprise one or more parts (b) comprising a series of medicaments comprising tapering doses of the opioid in compliance with step (2) of the dosage regimen described herein.

### EXAMPLE(S)

The invention will be further described by way of non-limiting example(s). It will be understood to persons skilled in the art of the invention that many modifications may be made without departing from the spirit and scope of the invention.

### Example 1—Preparation of Oral Formulation

Oral capsules are prepared from Cannabis extracts obtained by extraction of a Cannabis plant with ethanol, followed by removal of extractant by heating in vacuo. This results in a solid granule, which is divided into capsules having the constitution described in Table 1.

TABLE 1
Orally bioavailable capsules

| Ingredient | Amount per capsule |
|---|---|
| THC¹ | 50 mg |
| CBD¹ | 50 mg |
| Excipient(s) | qs 150 mg |

Notes:
¹The THC and CBD are obtained from extraction of a Cannabis plant using ethanol as extractant, which is removed by heating under reduced pressure, and each cannabinoid comprises up to about 6 wt % of a terpene fraction from the extraction process.

### Example 2—The Effectiveness of Medicinal Cannabis in a Rapid Opioid Dose Taper Regimen for Patients with Chronic Non-Cancer Pain

Twenty (20) patients with chronic non-cancer pain on a stable opioid medication regimen of daily morphine equivalent dose of >60 mg for more than 12 months are enrolled into a double blind, randomized, placebo-controlled Phase II study.

These patients are administered the capsule of Example 1 once daily, in addition to their opioid therapy. The opioid dosage is reduced over a period of 10 weeks by 10 wt % per week. This treatment period is followed by a period of 6 weeks follow-up where patients are administered neither cannabinoid or opioid therapy.

In addition to exploring the opioid sparing effects of medicinal cannabinoids and retention rates, this study also investigates the therapy's efficacy on improving patient mental health outcomes, well-being and cognition.

Amongst other endpoints, this study provides data supporting the use of cannabinoid-therapy as part of a rapid opioid dose taper regimen (opioid sparing) in patients suffering from chronic non-cancer pain. This is assessed during patient reviews prior to commencement of the trial and in Weeks 5, 10 and 16 of the study. Patient review at Week 5, Week 10 and Week 16 includes:
Time Line Follow Back—alcohol and tobacco use in preceding 30 days,
Self-reported psycho-social functioning using:
   Pain Self-efficacy questionnaire (PSEQ),
   Pain Catastrophizing Scale (PCS),
   K10 (Kessler Psychological Distress Scale),
   DASS21 (Depression, Anxiety and Stress Rating Scale, 21 questions), and/or
   ATOP Version 7.

The data collected during these scheduled reviews assess the following endpoints:
a. Change in opioid analgesia (OMEDD) from baseline at 5 weeks, 10 weeks, and 16 weeks (%).
b. Number of weeks until stepped dose taper is halted.
c. Retention in dose taper regimen at 5 weeks and 10 weeks.
d. Self-reported pain scores (Brief Pain Inventory)—daily.
e. Patient characteristics at baseline, 5, 10, and 16 weeks:
f. Pain Self-efficacy questionnaire (PSEQ)
g. Pain Catastrophizing Scale (PCS)
h. K10 (Kessler Psychological Distress Scale),
i. DASS21 (Depression, Anxiety and Stress Rating Scale, 21 questions),
j. ATOP (Australian Treatment Outcome Profile, version 7),
k. Urine profiles of cannabinoid metabolites (THC and its primary metabolite (THC-COOH) and CBD and its primary metabolite (CBD-7-oic acid)) (taken weekly).
l. Adverse events during the treatment period. This will be assessed by a self-report

Adverse Events Checklist administered by a clinician during assessment.

Each of the above endpoints a-j assesses the efficacy of the pharmaceutical composition of the invention in an opioid sparing protocol.

### Example 3—Preparation of Oromucosal Formulation

The liquid composition comprising 10 mg/mL THC and 10 mg/mL CBD was prepared from Cannabis extracts obtained by extraction of a Cannabis plant with ethanol, followed by removal of extractant by heating in vacuo. The extract was solubilized in olive oil. If required, THC and/or CBD or one or more terpenes/terpenoids may be added to the composition to provide the desired dosage.

The composition was packaged into pre-filled syringes containing the desired dosages.

### Example 4—Effectiveness of Oro-Mucosal Delivery of Cannabinoid Composition

Nine (9) patients with chronic non-cancer pain on a stable opioid medication regimen of daily morphine equivalent dose of >60 mg for more than 12 months are enrolled into the study.

These patients are administered the liquid formulation of Example 1, in addition to their opioid therapy in accordance with the dosage regimen below. All doses are split and administered morning and evening. The patients are to monitor their opioid usage over the study period.

### Dosage Regimen

Stage 1: Participants receive a single dose of composition containing 2.5 mg THC and 2.5 mg CBD followed by a 7 day wash out period.

Stage 2: Participants receive a single dose of composition containing 2.5 mg THC and 2.5 mg CBD followed by a high fat meal. Participants receive a total daily dose of a composition comprising 5 mg THC and 5 mg CBD for administration over the following week.

Stage 3: Participants receive a total daily dose of a composition comprising 10 mg THC and 10 mg CBD for administration over the following week.

Stage 4: Participants receive a total daily dose of a composition comprising 15 mg THC and 15 mg CBD for administration over the following 7 days.

Stage 5: Participants receive a single dose of composition containing 25 mg THC and 25 mg CBD followed by a 7 day wash out period.

The duration of the study is 36 days.

In addition to exploring the effects of medicinal cannabinoids and retention rates, this study also investigates the therapy's efficacy on improving patient mental health outcomes, well-being and cognition and the effects of a high fat diet.

In addition to the pharmacokinetic and safety parameters monitored, the effect parameters monitored include:
Brief Pain Inventory (BPI)
Depression, Anxiety and Stress Scale—21 questions (DASS-21)
Insomnia Severity Index (ISI) modified
Actigraphy—Sleep Onset Latency (SOL), Wake After Sleep Onset (WASO), Total Sleep Time (TST) and Sleep Efficiency (SE)
Sleep diary—total sleep time (sTST), time to fall asleep (sSOL), number of awakenings (sWASO), quality of sleep (sQual 5-point scale), refreshed feeling on wakening (sFRESH 5-point scale, rating of daytime energy/mood/functioning)
Self-reported opioid medication use

Each of the above endpoints assesses the efficacy of the pharmaceutical composition of the invention and the last point will assess the reduction in use of opioids over the study period.

1. A pharmaceutical composition comprising Δ⁹ tetrahydrocannabinol (THC), cannabidiol (CBD), a terpene fraction, and an opioid.

2. The pharmaceutical composition according to claim 1 comprising a Cannabis extract.

3. The pharmaceutical composition according to claim 1 wherein one of the following applies:
a) the ratio of THC:CBD is about 10:1 to about 1:10,
b) the ratio of THC:CBD is about 0.8:1 to about 1:2.1; or
c) the ratio of THC:CBD is about 1:1.

4. (canceled)

5. (canceled)

6. The pharmaceutical composition according to claim 1 wherein one or more of the following applies:
a) the THC is present in an amount of about 15 wt % to about 85 wt % of the composition;
b) the THC is present in an amount of about 15 wt % to about 40 wt % of the composition;
c) the CBD is present in an amount of about 15 wt % to about 60 wt % of the composition;
d) the CBD is present in an amount of about 15 wt % to about 40 wt % of the composition; and
e) the total amount of THC and CBD is in the range of about 30 wt % to about 99 wt %.

7. (canceled)

8. (canceled)

9. (canceled)

10. (canceled)

11. The pharmaceutical composition according to claim 1 comprising a Cannabis extract that is enriched in one and/or the other of THC and CBD.

12. The pharmaceutical composition according to claim 1 further comprising one or both of Cannabichromene (CBC) and Cannabigerol (CBG).

13. The pharmaceutical composition according to claim 1 wherein the terpene fraction is present in an amount of about 0.01 wt % to about 10 wt % of the composition.

14. The pharmaceutical composition according to claim 13 wherein one of the following applies:
a) the terpene fraction comprises one or more terpenes selected from the group consisting of β-myrcene, α-terpinene, linalool, α-phellandrene, camphene, terpinolene, p-cymene, 1,8-cineole, β-caryophyllene, d-limonene, γ-terpinene, α-pinene, guaiol, gurjunene, β-ocimene, β-pinene, γ-cadinene, caryophyllene oxide, nerolidol, and β-farnesene;
b) the terpene fraction comprises one or more terpenes selected from the group consisting of β-myrcene, α-terpinene, linalool, α-phellandrene, camphene, terpinolene, p-cymene, 1,8-cineole, and β-caryophyllene; or
c) the terpene fraction comprises one or more terpenes selected from the group consisting of β-myrcene, α-terpinene, linalool, and α-phellandrene.

15. (canceled)

16. (canceled)

17. (canceled)

18. The pharmaceutical composition according to claim 1 wherein the opioid is selected from the group consisting of oxycodone, hydrocodone, oxymorphone, morphine, codeine, fentanyl, buprenorphine, tramadol, and pethidine.

19. A method of opioid sparing, or treating opioid dependence and/or addiction, in a subject in need thereof, the method comprising administering to the subject an effective amount of the pharmaceutical composition of claim 1 according to the following dosage regimen:
(1) administering an effective amount of the pharmaceutical composition of claim 1 comprising an effective amount of the opioid, and
(2) administering an effective amount of the pharmaceutical composition of claim 1 comprising a sub-clinical amount of the opioid.

20. (canceled)

21. (canceled)

22. The method of any claim 19 wherein step (2) of the dosage regimen comprises reducing the dose of opioid by a tapering amount per week of treatment.

23. The method of claim 19 wherein the opioid is selected from oxycodone, hydrocodone, oxymorphone, morphine, codeine, fentanyl, buprenorphine, tramadol, pethidine.

24. The method of claim 19 wherein step (1) of the dosage regimen is maintained for a period of 1 to 10 weeks.

25. The method of claim 19 wherein step (2) of the dosage regimen is maintained for a period of 1 to 20 weeks.

26. (canceled)

27. (canceled)

28. The method according to claim 19 wherein the dosage of the composition of claim 1 is kept constant throughout the dosage regimen.

29. The method according to claim 19 wherein the dosage of the composition of claim 1 is varied throughout the dosage regimen.

30. The method according to claim 19 wherein the pharmaceutical composition is administered 1 to 4 times a day.

31. The method according to claim 19 wherein the subject or patient has chronic non-cancer pain.

32. (canceled)

33. (canceled)

34. (canceled)
